(19) United States
(12) Patent Application Publication
(10) Pub. No.: US 2010/0035955 A1
(43) Pub. Date: Feb. 11, 2010

(54) Stabilised Composition Comprising ACE Inhibitors

(76) Inventors: Panagiotis Keramidas, Carindale (AU); Brett Antony Mooney, Mt. Ommaney (AU); Sandra Jane Blundell, Forest Lake (AU)

Correspondence Address: SUMMA, ADDITON & ASHE, P.A., 11610 NORTH COMMUNITY HOUSE ROAD, SUITE 200, CHARLOTTE, NC, 28277, US

(21) Appl. No.: 12/307,029

(22) PCT Filed: Jun. 29, 2007
(86) PCT No.: PCT/AU2007/000907

§ 371 Date: Oct. 29, 2009

(30) Foreign Application Priority Data
Jun. 30, 2006 (AU) 2006903541

(51) Int. Cl. A61K 31/403; A61P 7/00

(52) U.S. Cl. 514/412

(57) ABSTRACT

A pharmaceutical composition comprising a) an ACE inhibitor prone to degradation or a pharmaceutically acceptable acid addition salt thereof; b) a stabilizing amount of an alkaline stabilizing agent; and c) pharmaceutically acceptable excipients wherein the composition further includes moisture controlling means.

The present invention relates to stabilized pharmaceutical compositions comprising ACE inhibitors.

### BACKGROUND

High blood pressure adds to the workload of the heart and arteries. If it continues for any length of time, the heart and arteries may not function properly. This can damage the blood vessels of the brain, heart, and kidneys and could result in a stroke, heart failure, or kidney failure. High blood pressure may also increase the risk of heart attacks. These problems may be less likely to occur if blood pressure is controlled.

Angiotensin Converting Enzyme (ACE) inhibitors belong to the class of medicines known as high blood pressure medicines or antihypertensives. They reduce the enzymatic conversion of angiotensin I to angiotensin II, which is a potent vasoconstrictor that causes blood pressure to increase. Many of the generic names for ACE inhibitors end in “pril”. Examples of ‘pril’ ACE inhibitors include Benazepril; Captopril; Cilazapril; Enalapril; Fosinopril; Lisinopril; Moexipril; Perindopril; Quinapril; Ramipril; Spirapril and Trandolapril

Ramipril, for example, is a 2-aza-bicyclo[3.3.0] octane-3-carboxylic acid derivative and is designated (2S,3aS,6aS)-1[(S)-N-[(S)-1-Carboxy-3-phenylpropyl]alanyl]octahydrocyclopenta[b]pyrrole-2-carboxylic acid, 1-ethyl ester. Its structural formula is:

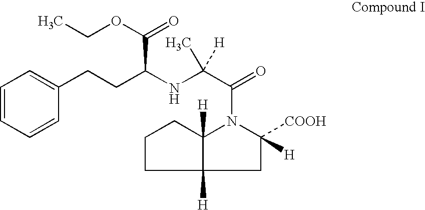

Ramipril is indicated for the treatment of hypertension. It may be used alone or in combination with thiazide diuretics.

It is also indicated for reducing the risk of myocardial infarction, stroke, cardiovascular death or need for revascularisation procedures in patients of 55 years or more who have clinical evidence of cardiovascular disease (previous myocardial infarction, unstable angina or multi-vessel CABG or multi-vessel PTCA), stroke or peripheral vascular disease.

It has been found that the ACE inhibitors and structurally related drugs are prone to degradation and show a tendency to be unstable when formulated into pharmaceutical compositions. The main decomposition products for ramipril are the diketopiperazine compound (II) produced by condensation, hereafter referred to as the diketo compound and the diacid compound (III), also known as ramiprilat. It has been found that the stability can be influenced by the choice of suitable excipients, and that a further significant cause of decomposition is the mechanical stress associated with the manufacturing process.

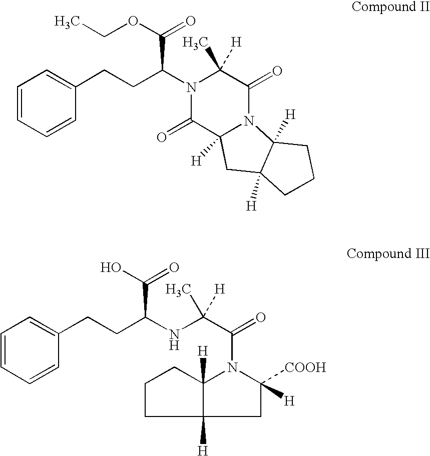

Corresponding degradation products are common to all the ‘pril’ ACE inhibitor compounds respectively.

Due to the beneficial properties of ACE inhibitors as antihypertensive agents there have been a number of attempts to overcome the associated instability problems.

For example, EP 264888 is directed to the stabilization of ACE inhibitor-containing pharmaceutical compositions employing ascorbic acid alone or a combination of ascorbic acid with fumaric acid, maleic acid and/or citric acid as the stabilizing component(s).

EP 468929 describes stabilization of ACE inhibitor compositions with a hydrochloric acid donor. Further applications relating to stabilized pharmaceutical compositions of ACE inhibitors comprise WO05041940 directed towards stabilization with meglumine, WO04071526 is directed towards dispersing a metal compound in alcohol and then mixing with the inhibitor.

U.S. Pat. Nos. 5,151,433 and 5,442,008 disclose polymer film-formers as protection against stress, as well as the use of buffers. The European equivalent, EP 317878 is further directed towards formulations comprising ACE inhibitors, stabilized by mixing the inhibitor with a buffer (excluding sodium bicarbonate) capable of maintaining the pH within the mildly acidic to mildly alkaline pH range, with the further proviso that in the case of alkali and alkaline-earth metal carbonates used as the buffer a sugar is not additionally incorporated into the formulation.

WO06050533 is directed towards individually coated, single ramipril crystalline particles and compositions comprising them.

U.S. Pat. No. 4,743,450 is directed to the stabilization of ACE inhibitor-containing pharmaceutical compositions, employing as the stabilizing component, a combination of an alkali or alkaline earth metal salt (preferably, magnesium carbonate) and a saccharide (preferably, mannitol or lactose).

WO9962560 is directed towards magnesium oxide as the stabilizing agent utilized in a composition comprising Quinapril and again a saccharide to prevent hydrolysis.

Thus, addition of either a stabilizer or a polymeric coat on the active ingredient is believed necessary to stabilize the pharmaceutical composition of ACE inhibitors, which are susceptible to degradation. However, the addition of some stabilizers can produce unwanted pharmacological effects. Coating the active ingredient is quite cumbersome, low yielding and moreover it requires specialized equipment.

The above prior art examples attempt to overcome the instability problems associated with ACE inhibitor-containing formulations. These prior art examples focus primarily on the stabilisation of the ACE inhibitor by reducing and/or preventing the formation of the diketo derivative. The examples of the reduction and/or prevention of the diacid derivative discuss the protection of the active by coating so as to minimise the influence of compression forces during tabletting and encapsulation. Of course, any degradation to one or both of these derivatives is undesirable as the shelf life of products containing such formulations is markedly reduced. Thus there still exists a need for alternative stabilised ACE inhibitor-containing compositions exhibiting improved stability during the formulation process and especially in the presence of moisture during extended periods of storage. To this end, the present invention is directed towards pharmaceutical compositions, particularly ACE inhibitor-containing compositions, exhibiting improved stability for both the diketo and diacid derivatives.

### SUMMARY OF INVENTION

Surprisingly it has been found by the inventors that a synergistic and not just additive effect is seen when the moisture levels in the immediate atmosphere surrounding a composition according to the invention are controlled and the stabilizing qualities of the stabilizing agent are combined, a more stable product results.

The addition of a stabilising agent to help control the formation of the diketo compound resulted in an increase in the level of diacid. Thus, the amount of stabilising agent used and the presence of the moisture control in the immediate atmosphere are critical in keeping the levels of both the diketo and the diacid at a suitably low level that does not appreciably increase over the shelf life of the product.

Accordingly there is provided a pharmaceutical composition comprising:
an ACE inhibitor prone to degradation or a pharmaceutically acceptable acid addition salt thereof;
a stabilizing amount of an alkaline-environment producing stabilizing agent; and
one or more pharmaceutically acceptable excipients,

further comprising means for controlling moisture levels.

In one preferred embodiment the stabilizing agent is magnesium oxide.

There is also provided a pharmaceutical kit comprising a sealable, moisture-impermeable container comprising a unit dosage form, wherein the unit dosage form comprises a pharmaceutical composition comprising:
an ACE inhibitor prone to degradation or a pharmaceutically acceptable acid addition salt thereof;
a stabilizing amount of an alkaline-environment producing stabilizing agent; and
one or more pharmaceutically acceptable excipients,
   further comprising means for controlling moisture levels within the sealed container.

Another embodiment of a kit according to the invention comprises the pharmaceutical excipients, lactose monohydrate, microcrystalline cellulose 101, magnesium oxide, microcrystalline cellulose 102, crospovidone and magnesium stearate.

Another aspect of the invention provides a pharmaceutical composition comprising:
an ACE inhibitor prone to degradation,
a stabilizing amount of an alkaline-environment producing stabilizing agent preferably magnesium oxide;
a polymer coating; and
one or more pharmaceutically acceptable excipients,
   further comprising means for controlling moisture levels.

A further aspect of the invention provides a pharmaceutical kit comprising a sealable, moisture-impermeable container comprising a unit dosage form comprising a pharmaceutical composition comprising:
an ACE inhibitor prone to degradation,
a stabilizing amount of an alkaline-environment producing stabilizing agent preferably magnesium oxide;
a polymer coating; and
one or more pharmaceutically acceptable excipients,
   further comprising means for controlling moisture levels within the sealed container.

Such pharmaceutical compositions of the invention can be made into unit dosage forms, as well known in the art of pharmaceutical manufacture. These dosage forms include tablets, capsules, suspensions and the like.

In yet another embodiment there is provided a pharmaceutical composition according to the invention wherein the unit dosage form comprises:

| | Percent w/w relative to the |
|---|---|
| Component | theoretical tablet weight (%) |
| ACE inhibitor | 1-80 |
| Lactose Monohydrate 100 # | 1-90 |
| Microcrystalline Cellulose 101 | 1-20 |
| Magnesium Oxide | 1-20 |
| Microcrystalline Cellulose 102 | 1-50 |
| Crospovidone | 1-20 |
| Magnesium Stearate | 1-20 |

Particularly preferred unit dosage forms according to the invention comprise:

| | Percent w/w relative to the |
|---|---|
| Component | theoretical tablet weight |
| Ramipril | 1-10% |
| Lactose Monohydrate 100 # | 55-65% |
| Microcrystalline Cellulose 101 | 10-15% |
| Magnesium Oxide | 1-5% |
| Purified Water | QS |
| Microcrystalline Cellulose 102 | 10-15% |
| Crospovidone | 1-10% |
| Magnesium Stearate | 1-5% |
| TOTAL (Theoretical Tablet Wt) | 100% |

In alternative embodiments the unit dosage form is coated preferably with a polymer coating such as the commercially available Opadry® coating system.

As mentioned above pharmaceutical kits according to the invention comprising both an alkaline-environment producing stabilizing agent and a means for controlling moisture content in the immediate atmosphere of the unit dosage form provides a more stable product compared to compositions comprising only stabilizing agents as found in the prior art. The enhanced stability can be seen in the reduced levels of the diketo compound found during stability tests. Tables 1-3 show stability testing results before and after a period of storage under conditions of 40° C. and 75% relative humidity. Table 1 shows the effect of the amount of stabilising agent on the formation of both diketo and diacid. Table 2 shows the effect of moisture control on the diacid levels of the product the subject of this invention. Table 3 shows the effect of the use of both the stabilising agent and moisture control on the diketo levels over prior art.

Accordingly in a preferred aspect of the invention there is provided a pharmaceutical composition comprising:
an ACE inhibitor prone to degradation or a pharmaceutically acceptable acid addition salt thereof;
a stabilizing amount an alkaline-environment producing stabilizing agent; and
one or more pharmaceutically acceptable excipients,
   further comprising means for controlling moisture levels, wherein the level of diketopiperazine impurity is between about 0 and 5% of the ACE-inhibitor content.

A further preferred aspect of the invention provides a pharmaceutical kit comprising a sealable, moisture-impermeable container comprising a unit dosage form, wherein the unit dosage form comprises a pharmaceutical composition comprising:
an ACE inhibitor prone to degradation or a pharmaceutically acceptable acid addition salt thereof;
a stabilizing amount an alkaline-environment producing stabilizing agent; and
one or more pharmaceutically acceptable excipients,

further comprising means for controlling moisture levels within the sealed container and wherein the level of diketopiperazine impurity is between about 0 and 5% of the ACE-inhibitor content.

Preferably the levels of the diketopiperazine impurity are between 0 and 2%, more preferably the levels are below 1 and most preferably the levels are below 0.5%.

In further embodiments the unit dosage form is preferably coated with a polymer coating

In further aspect according to the invention a pharmaceutical composition comprising:
an ACE inhibitor prone to degradation or a pharmaceutically acceptable acid addition salt thereof;
a stabilizing amount an alkaline-environment producing stabilizing agent; and
one or more pharmaceutically acceptable excipients,
   further comprising means for controlling moisture levels, wherein the ACE inhibitor is present at greater than 90% purity.

Preferably the purity of the ACE inhibitor is greater than 95%, most preferred is purity of greater than 97.5%.

In a still further aspect according to the invention a pharmaceutical kit is provided comprising a sealable, moisture-impermeable container comprising a unit dosage form, wherein the unit dosage form comprises a pharmaceutical composition comprising:
an ACE inhibitor prone to degradation or a pharmaceutically acceptable acid addition salt thereof;
a stabilizing amount an alkaline-environment producing stabilizing agent; and
one or more pharmaceutically acceptable excipients,
   further comprising means for controlling moisture levels within the sealed container and wherein the ACE inhibitor is present at greater than 90% purity.

Preferably the purity of the ACE inhibitor is greater than 95%, most preferred is purity of greater than 97.5%.

Preferably the ACE inhibitor is selected from the group comprising captopril, benazepril, enalapril, imidapril, lisinopril, fosinopril, ramipril, perindopril, quinapril, moexipril, and trandolapril, more preferably the ACE inhibitor is ramipril.

In further embodiments the unit dosage form is preferably coated with a polymer coating

### DETAILED DESCRIPTION

The invention provides a pharmaceutical composition and pharmaceutical kit made therefrom comprising a sealable, moisture-impermeable container comprising a unit dosage form, wherein said pharmaceutical composition comprises an ACE inhibitor prone to degradation or a pharmaceutically acceptable acid addition salt thereof, a stabilizing amount of an alkaline-environment producing stabilizing agent and one or more pharmaceutically acceptable excipients the kit further comprises means for controlling moisture levels within the sealed container. The unit dosage form comprises a stabilized formulation comprising ACE inhibitor's such as captopril, benazepril, enalapril, imidapril, lisinopril, fosinopril, ramipril, perindopril, quinapril, moexipril, and trandolapril as the active pharmaceutical ingredient. Preferably the stabilizing agent is a stabilizing amount of an alkaline-environment producing stabilizing agent preferably an alkali or alkaline metallic oxide, particularly preferred is magnesium oxide. Use of a stabilizing amount of an alkaline-environment producing stabilizing agent minimizes the degradation of ACE inhibitors and also improves the formulation of ACE inhibitors into pharmaceutical compositions by the wet granulation techniques. Other stabilizing agents that can be utilized in the working of this invention comprise any agent capable of providing alkaline conditions and as such may comprise alkali and alkaline earth metal salts for example carbonates, hydroxides and oxides, polymers, amino acids and the like.

Surprisingly it has been found by the inventors that a synergistic and not just additive effect is seen when the moisture levels in the immediate atmosphere surrounding a composition according to the invention is controlled and the stabilizing qualities of the stabilizing agent are combined.

TABLE 1
Effect of the amount of Stabilising
Agent on Diketo and Diacid Formation

| | | |
|---|---|---|
| Ratio of Active: | Stability Data: | Substance |

| Stabilising Agent | 40° C. + 75% RH | Diacid | Diketo |
|---|---|---|---|
| 1:4 | Initial | 0.892 | 0.007 |
| | 5 days | 7.362 | 0.023 |
| 1:0 | Initial | 0.000 | 0.072 |
| | 5 days | 0.516 | 1.091 |

The unit dosage form prepared from a composition according to the invention is stored under moisture controlling conditions to reduce exposure of said composition to atmospheric moisture. In certain embodiments of a kit according to the invention the sealable container comprises a container with a lid providing an airtight internal environment. In certain embodiments the moisture control means, preferably a desiccant, are contained within the lid and/or self-contained canister within the container.

In one embodiment of a kit according to the invention the means for controlling moisture within the sealed container comprises a desiccant located within the container. The container may be a bottle made of glass or any suitable material. The container may also comprise packaging with moisture-protective barriers such as cold-form film blister packs or blister packs containing desiccants in one or both films on one or both sides of the unit dosage form.

The surprisingly synergistic combination of alkaline environment and moisture control produces a unit dosage form that has reduced levels of compounds II and III that remain low even after extended storage periods, i.e. for the full shelf life of the product. The levels of both degradation products is significantly lower when both forms of product protection are employed concurrently than they both would be if only one form of protection was used.

TABLE 2
Effect of Moisture Control on Diacid Levels

Stability Data:
40° C. + 75% RH

| Diacid impurity (%) | Initial | 4 weeks |
|---|---|---|
| PVC/PVDC Blister Pack | 0.215 | 12.942 |
| Triplex Blister Pack | | 5.302 |
| Cold-form Foil Blister Pack | | 0.994 |
| HDPE Bottle, desiccant | | 0.391 |

It has also been found that the drying conditions used during manufacture to remove moisture effects the level of compound III. Increased time and/or temperature used during drying has increased the level of compound III. Therefore, traditional methods of moisture control during manufacture have not been found to be successful.

The synergistic combination of alkaline environment control and moisture control is effective even if the unit dosage form is a tablet that has been made by aqueous wet granulation techniques and/or aqueous coating.

The inventor's have further found that the amount of alkaline-environment producing stabilizing agent in a composition according to the invention can affect the stabilizing qualities of the composition. Due to the competing effect of the amount of stabilising agent on the levels of diketo and diacid, the level of stabilising agent must be optimised. Accordingly in pharmaceutical kits according to the invention, the ratio of stabilizing agent to ACE inhibitor is between 0.5:1-5:1. Preferably the ratio is about 1:1 to 2:1.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

It will be clearly understood that, although a number of prior art publications are referred to herein, this reference does not constitute an admission that any of these documents form part of the common general knowledge in the art, in Australia or in any other country.

### EXAMPLES

The following examples are merely illustrative of the present invention and they should not be considered as limiting the scope of the invention. For example it will be understood by one skilled in the art that the coating on the tablet formulations is an optional feature and in no way is suggested to limit the scope of the appended claims.

The following examples relate to a solid oral stabilized composition according to the invention. The composition comprises a tablet core which can be coated or uncoated. The tablets may be manufactured by any means available in the art but wet granulation is a particularly preferred method. The table below includes the coating ingredients but it is to understood that the coating is an optional embodiment. The tablets may be coated by any means available to the skilled person.

### Example 1

| | Percent w/w relative to the |
|---|---|
| Component | theoretical tablet weight |
| Ramipril | 2.5% |
| Lactose Monohydrate 100 # | 61.5% |
| Microcrystalline Cellulose 101 | 12.5% |
| Magnesium Oxide | 5% |
| Purified Water | QS |
| Microcrystalline Cellulose 102 | 12.5% |
| Crospovidone | 5% |
| Magnesium Stearate | 1% |
| TOTAL (Theoretical Tablet Core | 100% |
| Weight) | |
| Opadry ® &/or Opadry ® II Coating | 3% |
| Purified Water | N/A |
| TOTAL (Theoretical Coated | 101.5% |
| Tablet Weight) | |

### Example 2

| | Percent w/w relative to the |
|---|---|
| Component | theoretical tablet weight |
| Ramipril | 5% |
| Lactose Monohydrate 100 # | 59% |
| Microcrystalline Cellulose 101 | 12.5% |
| Magnesium Oxide | 5% |
| Purified Water | QS |
| Microcrystalline Cellulose 102 | 12.5% |
| Crospovidone | 5% |
| Magnesium Stearate | 1% |
| TOTAL (Theoretical Tablet Core | 100% |
| Weight) | |
| Opadry ® &/or Opadry ® II Coating¹ | 3.0% |
| Purified Water² | QS |
| TOTAL (Theoretical Coated | 103% |
| Tablet Weight) | |

### Example 3

This example shows the stability that the pharmaceutical kits according to the invention provide for the comprised unit dosage forms. It will of course be understood that unit dosage forms may comprise any solid oral dosage form such as tablets, capsules, mini-tablets, beads or pellets.

TABLE 3
Effect on Diketo Levels of both Stabilising
Agent & Moisture Control over Prior Art

| | | Stability Data: 40° C. + | | | |
|---|---|---|---|---|---|
| | | 75% RH | | | |
| Diketopiperazine | | | 4 | 8 | 12 |
| impurity (%) | Strength | Init. | weeks | weeks | weeks |
| Formulation according | 1.25 mg | 0.024 | 0.042 | 0.098 | 0.120 |
| to the invention | 2.5 mg | 0.012 | 0.036 | 0.067 | 0.087 |
| | 5 mg | 0.012 | 0.035 | 0.063 | 0.085 |
| Marketed formulation | 1.25 mg | 0.637 | 1.620 | 2.031 | 2.313 |
| | 2.5 mg | 0.402 | 0.927 | 1.212 | 0.957 |
| | 5 mg | 0.433 | 1.027 | 1.510 | 1.279 |

Of course it will be understood that the above examples are not intended to limit the scope if the invention. Those skilled in the art may make various changes and modifications without departing from the scope and spirit of the invention, which is defined in the claims below.

1. A pharmaceutical composition comprising
a) an ACE inhibitor prone to degradation or a pharmaceutically acceptable acid addition salt thereof;
b) a stabilizing amount of an alkaline stabilizing agent; and
c) pharmaceutically acceptable excipients
wherein the composition further includes moisture controlling means.

2. A pharmaceutical composition comprising
a) an ACE inhibitor prone to degradation wherein the ACE inhibitor is ramipril or a pharmaceutically acceptable acid addition salt thereof;
b) a stabilizing amount of an alkaline stabilizing agent; and
c) pharmaceutically acceptable excipients
wherein the composition further includes moisture controlling means.

3. A composition according to claim 1 wherein the alkaline stabilising agent is a metal oxide compound.

4. A composition according to claim 3 wherein the alkaline metal oxide compound is magnesium oxide.

5. A composition according to claim 1 wherein the moisture controlling means is selected from the group consisting of a desiccant and a moisture impermeable layer.

6. A composition according to claim 1 wherein the moisture controlling means is a desiccant.

7. A composition according to claim 1 wherein the ACE inhibitor prone to degradation comprises between about 1 to about 12 mg of ramipril.

8. A composition according to claim 7 comprising about 1.25 mg of ramipril.

9. A composition according to claim 7 comprising about 2.5 mg of ramipril.

10. A composition according to claim 7 comprising about 5 mg of ramipril.

11. A composition according to claim 7 comprising about 10 mg of ramipril.

12. (canceled)

13. A composition according to claim 1 further comprising the pharmaceutical excipients lactose monohydrate, microcrystalline cellulose 101, magnesium oxide, microcrystalline cellulose 102, Crospovidone and magnesium stearate.

14. (canceled)

15. (canceled)

16. (canceled)

17. A pharmaceutical composition comprising
a) an ACE inhibitor prone to degradation wherein the ACE inhibitor is ramipril or a pharmaceutically acceptable salt thereof,
b) a stabilizing amount of an alkaline metal oxide stabilizing agent; and
c) pharmaceutically acceptable excipients
wherein the composition further comprises a polymer coating.

18. (canceled)

19. (canceled)

20. (canceled)

21. A solid oral dosage form comprising the composition according to claim 1.

22. The dosage form according to claim 21 wherein the solid oral dosage form is a tablet or a capsule.

23. (canceled)

24. The dosage form according to claim 23 wherein the tablet is coated.

25. A pharmaceutical kit comprising the solid oral dosage form according to claim 21 wherein the solid oral dosage form is contained in a sealable, moisture-impermeable container and wherein said container further comprises means for controlling the moisture levels within the sealed container.

26. A pharmaceutical kit that includes a unit solid oral dosage form of a composition comprising:
a) an ACE inhibitor prone to degradation wherein the ACE inhibitor is ramipril or a pharmaceutically acceptable acid addition salt thereof;
b) a stabilizing amount of an alkaline stabilizing agent; and
c) pharmaceutically acceptable excipients,
further comprising means for controlling moisture levels within the sealed container] according to claim 21.

27-36. (canceled)

37. A pharmaceutical composition according to claim 13 comprising by weight:
ramipril in an amount of 2.5-5%;
lactose monohydrate 100 in an amount of 55-65%;
microcrystalline cellulose 101 in an amount of 10-15%;
Magnesium oxide in an amount of 2.5-5%;
microcrystalline cellulose 102 in an amount of 10-15%;
crospovidone in an amount of 2.5-10%; and
magnesium stearate in an amount of 0-5%;
wherein the weight percentages are based upon a theoretical tablet weight of 100%.

38. A pharmaceutical composition according to claim 37 comprising by weight:
ramipril in an amount of 2.5%;
lactose monohydrate 100 in an amount of 61.5%;
microcrystalline cellulose 101 in an amount of 12.5%;
Magnesium oxide in an amount of 5%;
microcrystalline cellulose 102 in an amount of 12.5;
crospovidone in an amount of 5%; and
magnesium stearate in an amount of 1.

39. A pharmaceutical composition according to claim 37 comprising by weight:
ramipril in an amount of 5%;
lactose monohydrate 100 in an amount of 59%;
microcrystalline cellulose 101 in an amount of 12.5%;
Magnesium oxide in an amount of 5%;
microcrystalline cellulose 102 in an amount of 12.5;
crospovidone in an amount of 5%; and
magnesium stearate in an amount of 1.

40. A pharmaceutical composition according to claim 17 comprising by weight:
ramipril in an amount of 2.5-5%;
lactose monohydrate 100 in an amount of 55-65%;
microcrystalline cellulose 101 in an amount of 10-15%;
Magnesium oxide in an amount of 2.5-5%;
microcrystalline cellulose 102 in an amount of 10-15%;
crospovidone in an amount of 2.5-10%;
magnesium stearate in an amount of 0-5%; and
a coating selected from the group consisting of Opadry and Opadry II in an amount of 0-5%;
wherein the weight percentages are based upon a theoretical tablet weight of 100%.

41. A pharmaceutical composition according to claim 17 comprising by weight:
ramipril in an amount of 2.5%;
lactose monohydrate 100 in an amount of 61.5%;
microcrystalline cellulose 101 in an amount of 12.5%;
Magnesium oxide in an amount of 5%;
microcrystalline cellulose 102 in an amount of 12.5;
crospovidone in an amount of 5%;
magnesium stearate in an amount of 1; and
a coating selected from the group consisting of Opadry and Opadry II in an amount of 3%.

42. A pharmaceutical composition according to claim 17 comprising by weight:
ramipril in an amount of 5%;
lactose monohydrate 100 in an amount of 59%;
microcrystalline cellulose 101 in an amount of 12.5%;
Magnesium oxide in an amount of 5%;
microcrystalline cellulose 102 in an amount of 12.5;
crospovidone in an amount of 5%;
magnesium stearate in an amount of 1; and
a coating selected from the group consisting of Opadry and Opadry II in an amount of 3%.
